(19) United States
(12) Patent Application Publication
(10) Pub. No.: US 2026/0253733 A1
(43) Pub. Date: Aug. 27, 2026

(54) AI-ASSISTED DIAGNOSTIC SUPPORT COMPANION PRODUCT PLATFORM

(71) Applicant: RSNA Ventures, LLC, Oak Brook, IL (US)

(72) Inventors: Deepa MADHAVAN, Oak Brook, IL (US); Karena GALVIN, Oak Brook, IL (US); Monish JOHN, Oak Brook, IL (US); Andrea SARTHER, Oak Brook, IL (US)

(21) Appl. No.: 19/551,352

(22) Filed: Feb. 26, 2026

Related U.S. Application Data
(60) Provisional application No. 63/763,467, filed on Feb. 26, 2025.

(51) Int. Cl. G16H 50/20; G06V 10/82; G10L 15/18; G16H 30/20; G16H 30/40; G16H 40/20; G16H 50/70

(52) U.S. Cl. CPC G16H 50/20; G06V 10/82; G10L 15/18; G16H 30/20; G16H 30/40; G16H 40/20; G16H 50/70

(57) ABSTRACT

A diagnostic support system for radiologists includes a user interface configured to receive user input and present relevant content, the user input comprising medical imaging data and speech input, an AI engine configured to process medical imaging data, a content matching engine comprising a relevance determinator and neural network, a content retrieval module configured to retrieve content from a multi-modal content database storing peer-reviewed resources, a speech recognition module, an image processing module, and an external systems interface configured to communicate with hospital information systems. The relevance determinator assesses significance and applicability of retrieved content, the neural network recognizes patterns and determines relevance, and the AI engine combines outputs to produce matched content comprising real-time diagnostic support presented via the user interface.

### CROSS-REFERENCE TO RELATED APPLICATION

This application claims priority to, and the benefit of, U.S. Provisional Patent Application No. 63/763,467, entitled AI-ASSISTED DIAGNOSTIC SUPPORT COMPANION PRODUCT PLATFORM, filed Feb. 26, 2025, which is hereby incorporated by reference in its entirety.

### TECHNICAL FIELD

The present disclosure relates to diagnostic support systems for radiologists, and more particularly to an AI-assisted product platform / system designed to provide real-time, relevant content from peer-reviewed content resources to assist radiologists during image interpretation. For purposes of this disclosure, the terms “system,” “platform,” and “product platform” may be used interchangeably.

### BACKGROUND

Radiologists interpret medical images to diagnose and monitor various conditions. As the volume and complexity of medical imaging data continue to grow, radiologists face challenges in efficiently analyzing and interpreting these images.

The field of radiology has seen significant increases in radiologists’ workloads, as well as in the complexity of patient cases. This increase in workload and case complexity has led to longer interpretation times and potential cognitive fatigue for radiologists. Additionally, the rapid pace of medical research and the continuous publication of new findings make it challenging to stay current with best practices.

Existing systems have limitations in providing radiologists with real-time relevant, peer-reviewed content at the point of care. Many current solutions lack the capability to efficiently filter and present up-to-date medical literature, case studies, and other relevant content in a manner that is both relevant to specific cases and easily digestible within the radiologist's workflow. This gap in content delivery can impact the speed and accuracy of diagnoses, particularly for complex or unusual cases.

### SUMMARY

According to an aspect of the present disclosure, a diagnostic support system for radiologists is provided. The system includes a user interface configured to receive user input and present relevant content to the user, where the user input comprises at least one of medical imaging data and speech input. The system further includes an AI engine configured to process medical imaging data, and a content matching engine configured to match the user input with the relevant content, the content matching engine comprising a relevance determinator and a neural network.

A content retrieval module is configured to receive matched content identifiers from the content matching engine and retrieve content from a multi-modal content database storing peer-reviewed content resources. The system also includes a speech recognition module configured to convert speech input to text, an image processing module configured to analyze medical images, and an external systems interface configured to communicate with hospital information systems. The relevance determinator assesses a significance and applicability of the retrieved content, the neural network recognizes patterns and makes determination as to a relevance of the retrieved content, and the AI engine combines outputs from the relevance determinator and the neural network to produce a final set of matched content from among the retrieved content, with the final set of matched content comprising real-time diagnostic support presented to the user via the user interface.

According to other aspects of the present disclosure, the diagnostic support system may include one or more of the following features. The AI engine may employ a hybrid approach combining computer vision, neural networks, and generative AI techniques for content matching and retrieval. The multi-modal content database may store medical journal articles, case studies, and imaging reference materials, and may be periodically updated to incorporate additional journal issues and published research. The speech recognition module may employ natural language processing algorithms to accurately interpret medical terminology and diverse speech patterns. The image processing module may be configured to process multiple imaging modalities including X-ray, computed tomography (CT), and magnetic resonance imaging (MRI), and may employ deep learning neural networks trained on large datasets of medical images to recognize patterns, anomalies, and specific features within the images.

The user interface may be configured to display a dashboard presenting an overview of current cases and highlighting those requiring immediate attention. The external systems interface may utilize standard healthcare data exchange protocols to ensure compatibility with a wide range of hospital systems, and may enable bidirectional communication with Picture Archiving and Communication Systems (PACS) and retrieve relevant patient data from Electronic Medical Record (EMR) systems.

According to another aspect of the present disclosure, a method for providing diagnostic support to radiologists is provided. The method includes receiving input data comprising at least one of medical imaging data or speech input and processing the input data using an AI engine. The method further includes querying a multi-modal content database storing peer-reviewed content resources based on the processed input data, and performing AI-assisted relevance ranking on query results by performing an initial assessment of potential content matches using a relevance determinator based on predefined criteria, conducting an in-depth analysis of the input data and the potential content matches using a neural network, and combining results from the relevance determinator and the neural network to produce a ranked set of matched content. The method also includes presenting the ranked set of matched content to a radiologist through a user interface, capturing feedback from the radiologist, and updating an AI model based on the captured feedback.

According to other aspects of the present disclosure, the method may include one or more of the following features. Processing the input data may comprise converting speech input to text using natural language processing algorithms configured to interpret medical terminology, and analyzing medical imaging data using deep learning neural networks trained on large datasets of medical images, including identifying and highlighting potential areas of concern within the medical images. Querying the multi-modal content database may comprise formulating a query based on extracted features from medical imaging data or converted text from speech input, and employing advanced database querying techniques to efficiently access and extract relevant content from a database storing medical journal articles, case studies, and imaging reference materials, which may be periodically updated to incorporate journal issues and published research.

Performing AI-assisted relevance ranking may comprise assessing retrieved content using machine learning algorithms and prioritizing content based on similarity to input data, recency, and applicability to the specific diagnostic context. Presenting relevant content to the radiologist may comprise displaying the content alongside original medical images in a user interface designed to integrate with the radiologist's workflow. Capturing feedback from the radiologist may comprise receiving assessments of the relevance and usefulness of the presented content and collecting additional insights or observations made by the radiologist. Updating the AI model based on the captured feedback may comprise refining relevance ranking algorithms, adjusting weighting of factors in the decision-making process, and expanding a knowledge base with new content derived from the radiologist's input.

The method may further comprise detecting, using the AI engine, one or more anomalies in the medical imaging data, determining that at least one detected anomaly corresponds to a potential diagnostic finding requiring immediate attention, generating an alert identifying the potential diagnostic finding, and automatically prioritizing a case in a radiologist's workflow queue based on the at least one detected anomaly. Processing the input data may also comprise converting the medical imaging data into a feature representation using an image processing neural network, generating embedding vectors from the feature representation representing semantic characteristics of the medical imaging data, partitioning the embedding vectors into clusters corresponding to different diagnostic categories, matching the clusters to content entries in the multi-modal content database, and synthesizing a content presentation comprising content entries corresponding to matched clusters tailored to the specific diagnostic context of the medical imaging data.

### BRIEF DESCRIPTION OF FIGURES

Non-limiting and non-exhaustive examples are described with reference to the following figures.

FIG. 1 illustrates a system diagram of a Diagnostic Support Companion Product Platform, according to aspects of the present disclosure.

FIG. 2 illustrates a system diagram of an AI-assisted Content Retrieval System, according to an embodiment.

FIG. 3 is a flowchart illustrating operations of an AI-Assisted Diagnostic Support Companion Product Platform for radiologists, in accordance with example embodiments.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure provides a new AI-Assisted Diagnostic Support Companion Product Platform for radiologists that integrates advanced artificial intelligence and machine learning technologies with existing radiological workflows and hospital information systems. This system leverages pattern recognition and provides decision support capabilities with seamless integration and operation.

A notable feature of the system described herein is its ability to provide real-time relevant, peer-reviewed content at the point of care. The system efficiently filters and presents up-to-date medical literature, case studies, and other relevant content in a manner that is both relevant to specific cases and easily digestible within the radiologist's workflow.

Furthermore, the system incorporates artificial intelligence technology, including a hybrid of computer vision, deep learning, and generative AI, to streamline and facilitate efficient content retrieval and rendering. These features are designed to handle medical terminologies and related content with improved accuracy and reliability, addressing ongoing challenges in the field and enhancing overall efficiency in radiological practice.

The Diagnostic Support Companion System described herein provides a novel approach to assisting radiologists in their diagnostic processes. This system integrates with radiological workflows to enhance the speed of diagnoses.

In some cases, the system may offer real-time peer-reviewed content resources at the point of care. These content resources may be semantically intelligent, leveraging AI to provide relevant content tailored to the specific case at hand. The system may be designed to seamlessly embed within the radiologist's existing workflow, thereby reducing the time spent on challenging or difficult-to-diagnose cases.

The Diagnostic Support Companion System may incorporate multi-modal search functionalities, including (without limitation) image-based and speech-based search functionalities. These features enable radiologists to interact with the system using multiple input modalities, thereby increasing the system's versatility and ease of use.

In some implementations, the system can support various medical imaging modalities. This broad support can enable the system to assist with a wide range of diagnostic tasks across different areas of radiology.

The system may also include mechanisms for continuous improvement. By capturing radiologist feedback and incorporating it into the AI model, the system may adapt and refine its performance over time.

While designed to enhance diagnostic processes, the Diagnostic Support Companion System may also prioritize patient privacy and data security. The system may be developed with consideration for existing compliance standards in medical data handling.

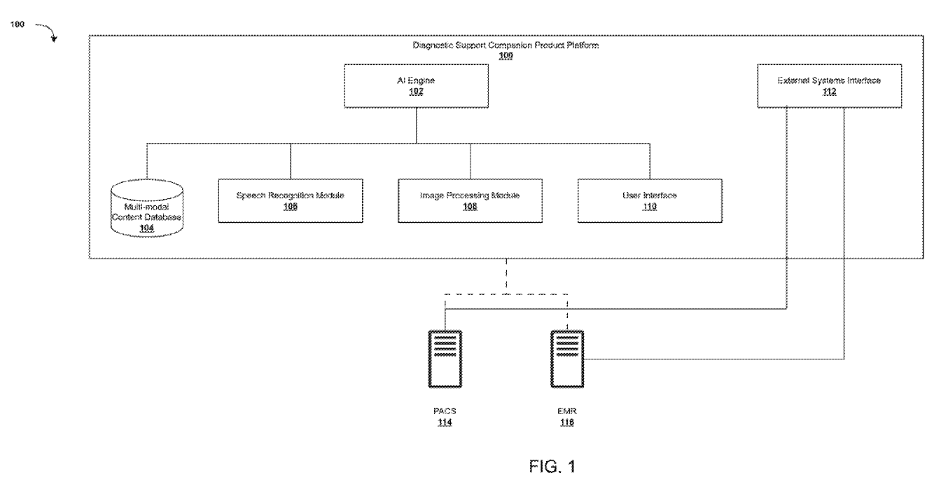

Turning now to FIG. 1, an exemplary Diagnostic Support Companion Product Platform 100 (also referred to as the ”system 100“) according to the present disclosure is shown. The system 100 illustrated in FIG. 1 represents one possible implementation for assisting radiologists in their diagnostic processes. It should be understood that other systems with different components and/or configurations are also possible within the scope of this disclosure. The system 100 in FIG. 1 integrates various components that may enhance the speed of diagnoses across multiple medical imaging modalities, though other arrangements according to this disclosure could provide similar functionality.

At the core of the system 100 is an AI Engine 102, which serves as the central processing unit for the Diagnostic Support Companion Product Platform 100. The AI Engine 102 can be configured to coordinate the operations of other components, as well as perform advanced analysis of medical data.

Connected to the AI Engine 102 is a Multi-modal Content Database 104. This database 104 can be configured to store a wide range of medical data, including peer-reviewed content resources, case studies, and other relevant content. The Multi-modal Content Database 104 supports the system's ability to provide relevant content for various types of medical imaging, including but not limited to X-ray, CT (Computed Tomography), and MRI (Magnetic Resonance Imagine).

The system 100 also incorporates a Speech Recognition Module 106 and an Image Processing Module 108, both linked to the AI Engine 102. These modules enable the system 100 to process different types of input. The Speech Recognition Module 106, for example, may be configured to convert spoken words into text, enabling radiologists to interact with the system 100 using voice commands. The Image Processing Module 108, on the other hand, can be configured to analyze medical images, supporting the system's capability to work with various imaging modalities.

A User Interface 110 is shown connected to the AI Engine 102, providing a means for radiologists to interact with the system 100. Through this interface 110, users may input data, view analysis results, and access relevant diagnostic content.

The Diagnostic Support Companion Product Platform 100 of FIG. 1 also includes an External Systems Interface 112. This interface 112 allows the system 100 to communicate with other medical systems utilized in healthcare settings. For example, the External Systems Interface 112 may connect to a Picture Archiving and Communication System (PACS) 114 and an Electronic Medical Record (EMR) system 116, just to name a few. These connections enable the Diagnostic Support Companion Product Platform 100 to integrate seamlessly with existing hospital infrastructure, enhancing its utility in clinical workflows.

By combining these components, the Diagnostic Support Companion Product Platform 100 may offer real-time, AI-assisted support for radiologists across a wide range of medical imaging modalities. The system's design allows for efficient processing of various inputs, access to relevant peer-reviewed content resources, and integration with existing medical systems, thereby improving the speed and accuracy of radiological diagnoses.

As noted above, the AI Engine 102 of the Diagnostic Support Companion Product Platform 100 can play a central role in processing and analyzing data from various sources to provide diagnostic support for radiologists. As a result, the AI Engine 102 can be configured to employ a sophisticated combination of artificial intelligence technologies to enhance the system's capabilities in content matching, retrieval and rendering.

In some cases, the AI Engine 102 can utilize a hybrid approach that combines computer vision, neural networks, and generative AI techniques. This multi-faceted approach enables the system 100 to handle diverse types of input and perform complex analyses on medical imaging data.

The computer vision component of the AI Engine 102, for example, may be configured to analyze and interpret medical images across various modalities. This capability enables the system 100 to identify relevant features, patterns, or anomalies within the images, which can be crucial for accurate diagnosis and efficient content retrieval and rendering.

Neural networks may be employed within the AI Engine 102 to process and learn from large volumes of medical data. These neural networks may be trained on extensive datasets of medical images, reports, and other relevant content, enabling the system 100 to recognize complex patterns and make informed decisions based on the input it receives.

The generative AI component of the AI Engine 102 may contribute to the system's ability to produce or synthesize new content based on existing data. This capability can be particularly useful in generating relevant content or suggestions for radiologists, enhancing the diagnostic support provided by the system 100.

In some cases, the generative AI capabilities of the AI Engine 102 may be used to synthesize concise summaries of relevant content or generate suggestions for further investigation based on the analyzed input and matched content.

The AI Engine's hybrid approach to content matching and retrieval may enable the Diagnostic Support Companion System to provide highly relevant and context-specific content to radiologists in real-time, thereby improving the speed and accuracy of diagnoses.

In some implementations, the AI Engine 102 may be configured to detect one or more anomalies in medical imaging data and determine whether a detected anomaly corresponds to a potential diagnostic finding requiring immediate attention. Upon detecting such an anomaly, the system may generate an alert identifying the potential diagnostic finding and automatically prioritize the case in the radiologist's workflow queue. This automatic prioritization may enhance diagnostic efficiency by directing the radiologist's attention to cases that may benefit most from immediate review, similar to how network security systems may automatically respond to detected threats.

The Multi-modal Content Database 104 serves as a comprehensive repository for various types of medical data, supporting the Diagnostic Support Companion System's 100 operations. This database 104 may store a wide range of content, including peer-reviewed content resources, medical literature, case studies, and other relevant content.

In some cases, the Multi-modal Content Database 104 may include user-specific or enterprise-specific peer-reviewed content resources such as RadioGraphics, an in-house journal publication. RadioGraphics represents just one example of a high-quality, curated source of radiological content that may be accessed by the system 100 to provide relevant diagnostic support.

The database 104 can be structured to accommodate different types of content, including text, images, audio or video files, etc. This multi-modal approach enables the system 100 to retrieve and present content in various formats, depending on the specific needs of the radiologist and the nature of the diagnostic task at hand.

To support efficient retrieval and relevance matching, the Multi-modal Content Database 104 can be configured to employ advanced indexing and tagging systems, for example. These systems can allow for rapid (multi-modal) searching and filtering of content based on various criteria such as imaging modality, anatomical region, pathology, specific diagnostic features, and so on.

In some cases, the database 104 can be regularly updated to ensure that the content remains current and reflects the latest advancements in radiological knowledge. This updating process can involve the addition of new journal issues, such as the latest edition of RadioGraphics, the incorporation of newly published research and content, and the like.

The Multi-modal Content Database 104 can also be configured to support the AI-assisted aspects of the Diagnostic Support Companion Product Platform 100. For example, the database's structure may facilitate machine learning processes, enabling the system 100 to improve its content matching and relevance determination capabilities over time.

The Diagnostic Support Companion Product Platform 100 also includes a Speech Recognition Module 106 that enables speech-based search functionality. This module 106 can be configured to process audio inputs and convert spoken words into text format, enabling radiologists to interact with the system 100 using voice commands. In some embodiments, the speech-based search functionality of module 106 can be combined with and/or cooperate with image-based search functionality (e.g., as in module 108, discussed below) to provide multi-modal search functionalities.

In some cases, the Speech Recognition Module 106 can employ advanced natural language processing algorithms to accurately interpret medical terminology and diverse speech patterns. The module 106 can also be configured to handle various accents, speaking speeds, speech patterns, and background noise levels commonly encountered in clinical settings.

The Speech Recognition Module 106 can operate by first capturing audio input through a microphone or other audio input device. The captured audio can then be processed to filter out background noise and enhance the clarity of the speech signal. Following this initial processing, the module 106 can apply speech recognition algorithms to convert the audio into text.

In some implementations, the Speech Recognition Module 106 can utilize machine learning techniques to improve its accuracy over time. The module 106 can be trained on large datasets of medical speech to enhance its ability to recognize and accurately transcribe specialized medical terminology and phrases commonly used in radiology.

Once the speech input has been converted to text, the system 100 can treat this text as a search query. The converted text can be passed to other components of the system 100 for further processing and analysis, enabling the system 100 to perform text-based searches of its database 104 and retrieve relevant content.

The Speech Recognition Module 106 can also be configured to support various types of voice commands and queries. For example, radiologists may be able to request content about specific conditions, ask for similar case studies, and/or initiate searches for particular imaging features using voice commands.

In some cases, the Speech Recognition Module 106 can be configured to work in conjunction with other components of the system 100 to provide a seamless user experience. For instance, this module 106 can integrate with the user interface 110 (discussed below) to display the recognized text in real-time, allowing users to verify the accuracy of the speech recognition and make corrections if necessary.

The inclusion of multi-modal searching (e.g., a combination of speech-based search functionality alongside image-based searching) provides radiologists with flexible options for interacting with the system, potentially improving efficiency and ease of use in various clinical scenarios.

The Image Processing Module 108 of the Diagnostic Support Companion Product Platform 100 can be configured to analyze and interpret medical images across various modalities. This module 108 can employ advanced computer vision techniques and machine learning algorithms to process and extract relevant content from medical imaging data. As noted above, the image-based search functionality of this module 108 can be combined with and/or cooperate with speech-based search functionality (e.g., as in module 106, discussed above) to provide multi-modal search functionalities.

In some cases, the Image Processing Module 108 can be configured to process a wide range of imaging modalities, including but not limited to X-ray, computed tomography (CT), magnetic resonance imaging (MRI), ultrasound, and positron emission tomography (PET) scans. This versatility enables the system 100 to provide diagnostic support across different areas of radiology.

The Image Processing Module 108 can utilize deep learning neural networks trained on large datasets of medical images to recognize patterns, anomalies, and specific features within the images. These neural networks may be designed to identify and highlight potential areas of concern, such as tumors, fractures, or other abnormalities, depending on the specific imaging modality and anatomical region being examined.

In some implementations, the Image Processing Module 108 can incorporate segmentation algorithms to delineate different anatomical structures within the images. This segmentation capability can assist in isolating specific regions of interest for more detailed analysis or comparison with reference images.

This module 108 can also leverage feature extraction algorithms that can identify and quantify specific characteristics of the images, such as tissue density, texture, or shape. These extracted features can be used to support the diagnostic process by providing quantitative data that can be compared against established norms or used in conjunction with other clinical content.

In some cases, the Image Processing Module 108 can employ image registration techniques to align and compare multiple images, either from the same patient over time or between different patients. This capability may be particularly useful for tracking the progression of conditions or identifying subtle changes that may not be immediately apparent to the human eye.

The Image Processing Module 108 may also utilize noise reduction and image enhancement algorithms to improve the quality and clarity of the medical images. These preprocessing steps may help to highlight important details and make the images more suitable for both automated analysis and human interpretation.

In some implementations, the Image Processing Module 108 may be configured to generate three-dimensional (3D) reconstructions from two-dimensional image slices, particularly for modalities such as CT and MRI. These 3D reconstructions may provide additional insights into the spatial relationships of anatomical structures and pathologies.

The Image Processing Module 108 can also work in conjunction with other components of the Diagnostic Support Companion Product Platform 100 to provide a comprehensive analysis of medical images. For example, the results of the image processing may be used to query the Multi-modal Content Database 104 for relevant case studies or literature that match the identified features or patterns.

In some cases, the Image Processing Module 108 may incorporate natural language processing capabilities to analyze and interpret text annotations or reports associated with the medical images. This feature can enable the system 100 to correlate textual content with visual data, enhancing the overall diagnostic support provided.

This module 108 may also include mechanisms for continuous learning and improvement. As new imaging data and validated diagnoses are added to the system 100, the Image Processing Module 108 can update and refine its algorithms to improve accuracy and performance over time.

In some implementations, the Image Processing Module 108 can be configured to generate heatmaps or other visual overlays to highlight areas of potential concern within the images. These visual aids may help guide radiologists' attention to specific regions that warrant closer examination.

The Image Processing Module 108 can also be configured with consideration for computational efficiency, allowing for rapid processing of large volumes of imaging data. This efficiency can further support real-time analysis and the providing of timely diagnostic support in clinical settings.

In some cases, the module 108 can include specialized algorithms for detecting and classifying specific pathologies or conditions, such as lung nodules in chest X-rays or brain lesions in MRI scans. These targeted algorithms can be developed and refined based on the latest research and clinical standards in various subspecialties of radiology.

The Image Processing Module 108 can also incorporate mechanisms for explaining its analysis results, providing radiologists with insights into how the system 100 arrived at its conclusions. This explainability feature can further enhance trust in the system 100 and support the radiologist's decision-making process.

In some implementations, the Image Processing Module 108 can also convert medical imaging data into a feature representation using an image processing neural network. The system 100 can then generate embedding vectors from the feature representation, wherein the embedding vectors represent semantic characteristics of the medical imaging data. These embedding vectors may be partitioned into clusters corresponding to different diagnostic categories. The clusters can then be matched to content entries in the Multi-modal Content Database 104. The system 100 can synthesize a content presentation comprising content entries corresponding to matched clusters, wherein the content presentation is tailored to the specific diagnostic context of the medical imaging data. This approach can enable the system 100 to provide highly relevant content even when the specific diagnostic features have not been previously encountered.

The Diagnostic Support Companion Product Platform 100 further includes a User Interface 110 configured to provide a means for radiologists to interact with the system 100. This User Interface 110 may be designed to facilitate efficient input of data and presentation of diagnostic support content.

In some cases, the User Interface 110 may incorporate a graphical user interface (GUI) that displays relevant content in a visually intuitive manner. The GUI may include various elements such as windows, menus, buttons, and text fields to enable user interaction.

The User Interface 110 can be configured to accept multiple types of input from radiologists. For example, the User Interface 110 may allow for text input through a keyboard, voice input through a microphone, or image input through file uploads or direct connections to imaging devices.

In some implementations, the User Interface 110 can include a dashboard that presents an overview of current cases, highlighting those that require immediate attention or have been flagged by the system 100 as potentially challenging. This dashboard may help radiologists prioritize their workload and focus on cases that may benefit most from the system's diagnostic support.

The User Interface 110 can also provide a means for radiologists to view and interact with medical images. This may include tools for zooming, panning, adjusting contrast, and applying various image processing filters to enhance visibility of specific features.

In some cases, the User Interface 110 can display the results of the system's analysis alongside the original medical images. This may include highlighting areas of potential concern, providing measurements, or overlaying additional content derived from the system's processing.

The User Interface 110 can be configured to also present relevant content retrieved from the system's database 104 in a structured and easily digestible format. This may include summaries of similar cases, excerpts from relevant literature, content pertinent to the current case, etc.

In some implementations, the User Interface 110 can include interactive elements that enable radiologists to provide feedback on the system's performance. This may involve rating the relevance of presented content, confirming or rejecting suggested diagnoses, or annotating images with additional observations.

The User Interface 110 can further be configured with consideration for the specific workflow of radiologists. For example, it may support rapid navigation between different cases, easy access to patient history and previous imaging studies, and efficient report generation tools.

In some cases, the User Interface can be customizable, enabling individual radiologists to adjust the layout, content density, or color scheme according to their preferences. This customization may help improve user comfort and efficiency when working with the system 100 over extended periods.

The User Interface 110 can also incorporate notification systems to alert radiologists of important updates or time-sensitive content. These notifications can be configurable to avoid unnecessary interruptions while ensuring that critical content is promptly communicated.

In some implementations, the User Interface 110 can be configured to support multi-monitor setups, enabling radiologists to distribute different aspects of the interface across multiple screens for improved visibility and workflow management.

The User Interface 110 can also include features to support collaboration among healthcare professionals. This may involve tools for sharing cases, annotating images, or discussing findings with colleagues, all within the secure environment of the system 110.

In some cases, the User Interface 110 can provide access to educational content resources or training modules, enabling radiologists to enhance their skills.

The User Interface 110 can also be configured with accessibility considerations in mind, including features such as adjustable text sizes, high-contrast modes, or screen reader compatibility to accommodate users with different preferences or needs.

In some implementations, the User Interface 110 can include a search function that enables radiologists to quickly locate specific cases, patients, or content within the system's database 104.

The User Interface 110 may also provide visualization tools for presenting statistical data or trends derived from the system's analysis of multiple cases. This can be used to assist radiologists in identifying patterns or correlations that may not be apparent when examining individual cases in isolation.

The Diagnostic Support Companion Product Platform 100 of FIG. 1 can also include an External Systems Interface 112 that enables communication and data exchange with existing hospital information systems. This interface 112 may facilitate integration with Picture Archiving and Communication Systems (PACS) 114, Electronic Medical Record (EMR) systems 116, and/or other systems that may be utilized in healthcare settings.

In some cases, the External Systems Interface 112 can utilize standard healthcare data exchange protocols to ensure compatibility with a wide range of hospital systems. These protocols may include, for example, Health Level 7 (HL7) for exchanging clinical and administrative data, or Digital Imaging and Communications in Medicine (DICOM) for handling medical imaging content.

The External Systems Interface 112 can be configured to retrieve relevant patient data from connected EMR systems 116. This capability may enable the Diagnostic Support Companion Product Platform 100 to access patient history, previous diagnoses, and other clinical content that may be pertinent to the current radiological examination.

In some implementations, the External Systems Interface 112 may enable bidirectional communication with PACS 114. This can enable the Diagnostic Support Companion Product Platform 100 to retrieve medical images for analysis and potentially store processed results or annotations back into the PACS 114 for future reference.

The External Systems Interface 112 can also incorporate security measures to ensure the confidentiality and integrity of patient data during transmission between systems. These measures may include, for example, encryption protocols and authentication mechanisms to comply with healthcare data protection regulations.

In some cases, the External Systems Interface 112 can be configured to support real-time data synchronization between the Diagnostic Support Companion Product Platform 100 and connected hospital systems. This feature may be leveraged to ensure that radiologists have access to the most up-to-date patient information and imaging studies during their diagnostic processes.

The External Systems Interface 112 can also include mechanisms for handling different data formats and structures used by various hospital systems. This flexibility can allow the Diagnostic Support Companion Product Platform 100 to integrate seamlessly with diverse healthcare IT environments.

In some implementations, the External Systems Interface 112 can provide logging and auditing capabilities to track data exchanges between systems. This feature can be useful in troubleshooting, system optimization, and compliance with healthcare regulations regarding data access and usage.

The External Systems Interface 112 can be designed with scalability in mind, allowing for the addition of new connections to other hospital systems or external databases as needed. This extensibility can enable the Diagnostic Support Companion Product Platform 100 to adapt to evolving healthcare IT landscapes and incorporate new data sources over time.

As discussed above, the Diagnostic Support Companion Product Platform 100 shown in FIG. 1 integrates various components to provide comprehensive diagnostic support for radiologists. The system's architecture facilitates seamless interaction between its modules, enabling efficient processing of inputs, analysis of medical data, and retrieval and rendering of relevant content.

The Diagnostic Support Companion Product Platform 100 can also include mechanisms for monitoring and validating the system's performance. This can include validating the accuracy and/or relevance of any AI-generated responses. In some implementations, human experts can review and validate the system's outputs before they are released to users. Such human oversight can serve as an additional quality control measure, helping to maintain the integrity and trustworthiness of the system 100. This validation process can help ensure the accuracy and reliability of the diagnostic support provided by the system 100.

To measure improvements in diagnosis speed, the system 100 can track case turnaround time. In some cases, this can involve measuring the time from when a radiologist first views an image to when the final report is signed off. This metric can provide insights into the system's effectiveness in supporting radiologists and potentially reducing the time required for challenging or difficult-to-diagnose cases.

The integration and interaction of the aforementioned components enables the Diagnostic Support Companion Product Platform 100 to provide timely, relevant, and validated diagnostic support to radiologists, thereby improving the efficiency and accuracy of their work across a wide range of medical imaging modalities.

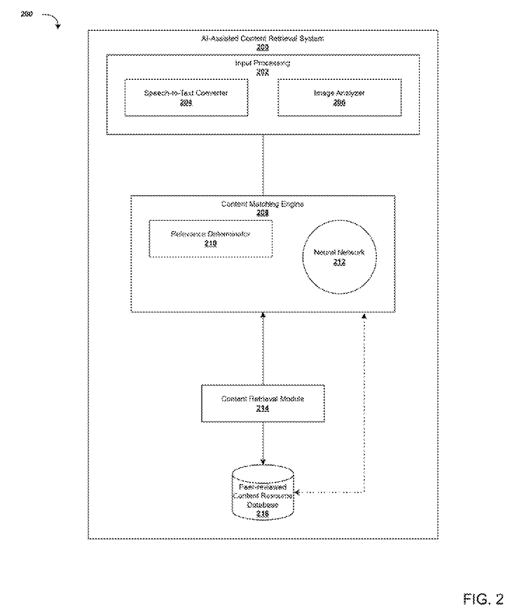

Turning now to FIG. 2, an AI-Assisted Content Retrieval System 200, which may be a component (e.g., a sub-system) of the Diagnostic Support Companion Product Platform 100 described above, is shown. The Content Retrieval System 200 can be configured to efficiently process various inputs, match them with relevant content, and retrieve appropriate content to support radiologists in their diagnostic tasks.

In some cases, the AI-Assisted Content Retrieval System 200 can employ a sophisticated hybrid approach that combines computer vision, neural networks, and generative AI technologies for content matching and retrieval, as discussed above. This multi-faceted approach can enable the system 200 to handle diverse types of input and perform complex analyses on medical imaging data.

The AI-Assisted Content Retrieval System 200 shown in FIG. 2 includes an Input Processing component 202 configured to handle incoming data from various sources. This component 202, as shown, comprises two main subcomponents: a Speech-to-Text Converter 204 and an Image Analyzer 206. These subcomponents enable the system 200 to process different types of input, providing flexibility for radiologists in their interactions with the system.

The Speech-to-Text Converter 204 can be configured to handle audio input, converting spoken words into text format. In some cases, this converter 204 can employ advanced natural language processing algorithms to accurately interpret medical terminology and diverse speech patterns. For example, a radiologist may speak a description of an observed anomaly, and the Speech-to-Text Converter 204 can in turn transform this spoken input into a textual format suitable for further processing and analysis by the system 200.

The Image Analyzer 206 can be designed to process visual input, particularly medical images across various modalities. In some implementations, the Image Analyzer 206 can utilize computer vision techniques and machine learning algorithms to extract relevant features and content from the input images. For instance, when presented with an X-ray image, the Image Analyzer 206 can identify and highlight specific anatomical structures or potential areas of concern.

The Input Processing component 202 can support a wide range of medical imaging modalities. In some cases, this component 202 can be configured to process inputs from X-ray, computed tomography (CT), magnetic resonance imaging (MRI), and other imaging technologies. This versatility enables the system 200 to provide comprehensive support across different areas of radiology.

In some implementations, the Input Processing component 202 can be designed to operate in conjunction with other parts of the AI-Assisted Content Retrieval System 200 to provide a seamless user experience. For example, after the Speech-to-Text Converter 204 transforms spoken input into text, this text may be used as a basis for a text-based search within the system's database. 216 Similarly, the output from the Image Analyzer 206 can be used to query the system 200 for relevant case studies or literature that match the identified features or patterns in the input image.

The Input Processing component 202 may also incorporate mechanisms for preprocessing and standardizing inputs. In some cases, this may involve noise reduction techniques for audio inputs or image enhancement algorithms for visual inputs. These preprocessing steps can be executed to improve the accuracy and reliability of subsequent analysis and content retrieval operations.

The AI-Assisted Content Retrieval System 200 also includes a Content Matching Engine 208 configured to match input data with relevant content. The Content Matching Engine 208, in this example, comprises two main subcomponents: a Relevance Determinator 210 and a Neural Network 212. These subcomponents can work in tandem to analyze input data and identify the most pertinent content for radiologists.

The Relevance Determinator 210 can be configured to assess the significance and applicability of potential matches based on various criteria. To that end, the Relevance Determinator 210 can employ algorithms that consider factors such as the similarity of image features, the context of the current case, and the specificity of the input query. This component 210 may also be configured to filter and prioritize potential matches, ensuring that only the most relevant content is presented to the radiologist.

The Neural Network 212 can be configured to process complex patterns and relationships within the input data and the available content. In some implementations, the Neural Network 212 can be trained on large datasets of medical images, reports, and other relevant content. This training can enable the Neural Network 212 to recognize intricate patterns and make informed decisions about content relevance.

The Content Matching Engine 208 can operate by first receiving processed input from the Input Processing component 202. This input can be in the form of extracted features from medical images or converted text from speech input. The Relevance Determinator 210 can then perform an initial assessment of potential matches based on predefined criteria and heuristics.

Following this initial assessment, the Neural Network 212 can conduct a more in-depth analysis of the input and potential matches. In some cases, the Neural Network 212 can leverage its trained models to identify subtle correlations and similarities that may not be immediately apparent through traditional matching algorithms.

The results from both the Relevance Determinator 210 and the Neural Network 212 can be combined to produce a final set of matched content. This combination can involve weighting the outputs of each subcomponent based on various factors, such as the confidence levels of their respective assessments.

In some implementations, the Content Matching Engine 208 can employ an iterative process, refining its matches based on feedback from other components of the system 200. For example, if the Content Retrieval Module 214 indicates that certain matched content is not available or accessible, the Content Matching Engine 208 can adjust its matching criteria and produce alternative recommendations.

The Content Matching Engine 208 can also be configured to adapt and improve its performance over time. In some cases, the system 200 can incorporate feedback from radiologists about the relevance and usefulness of presented content. This feedback can be used to fine-tune the algorithms of the Relevance Determinator 210 and update the training of the Neural Network 212, leading to more accurate and helpful matches in future operations.

By leveraging artificial intelligence techniques within the Content Matching Engine 208, the system 200 can determine the most relevant content to present to radiologists. This AI-assisted approach enables the system 200 to provide tailored, context-specific support that aligns closely with the needs of each individual case and radiologist.

The AI-Assisted Content Retrieval System 200 includes a Content Retrieval Module 214 that contributes to the accessing and delivering of relevant content to support radiologists in their diagnostic tasks. This module 214 can be configured to interact closely with other components of the system 200, including the Content Matching Engine 208 and the Peer-reviewed Content Resource Database 216.

The Content Retrieval Module 214 can be configured to receive input from the Content Matching Engine 208, which provides a set of matched content based on the processed input data. In some cases, the Content Retrieval Module 214 can use this content to query the Peer-reviewed Content Resource Database 216 and retrieve the specific content identified as relevant.

In some implementations, the Content Retrieval Module 214 can employ advanced database querying techniques to efficiently access and extract the required content from the Peer-reviewed Content Resource Database 216. This can involve optimized search algorithms and indexing methods to minimize retrieval time and ensure rapid delivery of relevant content to the user.

The Content Retrieval Module 214 can also be configured for formatting and organizing the retrieved content for presentation to the radiologist. In some cases, this may involve summarizing lengthy articles, extracting key passages, or compiling relevant content from multiple sources into a cohesive format.

In some implementations, the Content Retrieval Module 214 can incorporate caching mechanisms to store frequently accessed content locally. This feature can help reduce the load on the Peer-reviewed Content Resource Database 216 and improve system 200 response times for commonly requested content.

The Content Retrieval Module 214 can also be configured to handle various types of content, including text, images, and multimedia content resources, for example. In some cases, the module 214 can be configured to process and prepare these different content types for seamless integration into the user interface.

In some implementations, the Content Retrieval Module 214 can include error handling and fallback mechanisms. For instance, if a piece of matched content is unavailable or inaccessible in the Peer-reviewed Content Resource Database 216, the module 214 can be configured to notify the Content Matching Engine 208 to provide alternative recommendations.

The Content Retrieval Module 214 can also be configured with scalability in mind, capable of handling increasing volumes of data and concurrent requests as the system's usage grows. This may involve implementing load balancing techniques and optimizing database connections to maintain performance under varying workloads.

In some cases, the Content Retrieval Module 214 may incorporate logging and analytics capabilities. These features may help track usage patterns, identify popular content, and provide insights that can be used to improve the overall performance and relevance of the content retrieval process.

The Content Retrieval Module 214 can also be configured for enforcing access controls and ensuring that retrieved content adheres to any applicable copyright or licensing restrictions. This can involve checking user permissions and applying appropriate content filters based on predefined rules or policies.

The Content Retrieval Module 214 can also be configured to handle real-time updates to the Peer-reviewed Content Resource Database 216. In some cases, the module 214 can be configured to incorporate newly added or updated content into its retrieval processes without requiring system downtime or manual intervention.

The Peer-reviewed Content Resource Database 216 discussed above can serve as a comprehensive repository for verified medical content. This database 216 can store a wide range of content resources to support the system's functionality in providing relevant and reliable content to radiologists.

In some cases, the Peer-reviewed Content Resource Database 216 can contain various types of content, including medical journal articles, case studies, imaging reference materials, and other relevant content. These content resources may be curated to ensure their relevance and reliability in supporting radiological diagnoses.

The Peer-reviewed Content Resource Database 216 can also include enterprise-specific peer-reviewed content resources such as RadioGraphics, a journal publication that focuses on radiological topics. RadioGraphics may represent one example of a high-quality, curated source of content that the system 200 can access to provide relevant diagnostic support, as noted above.

In some implementations, the Peer-reviewed Content Resource Database 216 can be structured to accommodate different types of content, including text, images, multimedia content resources, and others. This multi-modal approach enables the system 200 to retrieve and present content in various formats, depending on the specific needs of the radiologist and the nature of the diagnostic task at hand.

The Peer-reviewed Content Resource Database 216 can be regularly updated to ensure that the content remains current and reflects the latest advancements in radiological knowledge. In some cases, this updating process can involve periodic refreshes of journal contents, such as the addition of new issues of RadioGraphics or other relevant publications.

To support efficient retrieval and relevance matching, the Peer-reviewed Content Resource Database 216 can employ advanced indexing and tagging systems. These systems allow for rapid searching and filtering of content based on various criteria such as imaging modality, anatomical region, pathology, or specific diagnostic features.

In some implementations, the Peer-reviewed Content Resource Database 216 can be configured to support the AI-assisted aspects of the Content Retrieval System 200. The database's structure can facilitate machine learning processes, thereby enabling the system 200 to improve its content matching and relevance determination capabilities over time.

In some cases, the Peer-reviewed Content Resource Database 216 can include mechanisms for handling different levels of access or subscription-based content. This can be used to ensure that users only retrieve content they are authorized to access, while still providing a comprehensive range of content resources to support diagnostic processes.

The Peer-reviewed Content Resource Database 216 can be configured with scalability in mind, capable of accommodating growing volumes of data as new research is published. This scalability can help ensure that the Content Retrieval System 200 remains up-to-date and comprehensive in its support for radiological diagnoses.

In operation, the system 200 can begin with input processing, where the Input Processing component 202 analyzes and converts speech and/or image data into a format suitable for further processing. The processed input can then be passed to the Content Matching Engine 208 for analysis and relevance determination.

The Content Matching Engine 208 can employ artificial intelligence techniques to identify the most relevant content based on the input. In some implementations, this can involve using neural networks and other machine learning algorithms to recognize patterns and make informed decisions about content relevance.

Once relevant content has been identified, the Content Retrieval Module 214 can access the Peer-reviewed Content Resource Database 216 to fetch the specific content. This retrieved content may then be formatted and presented to the radiologist through a user interface.

In some cases, the system 200 can incorporate a feedback loop where radiologists can provide input on the relevance and usefulness of the presented content. This feedback may be used to refine and improve the system's performance over time.

The integration of the foregoing components and processes into the AI-Assisted Content Retrieval System 200 can enable the system 200 to provide timely, relevant, and validated diagnostic support to radiologists, thereby improving the efficiency and accuracy of their work.

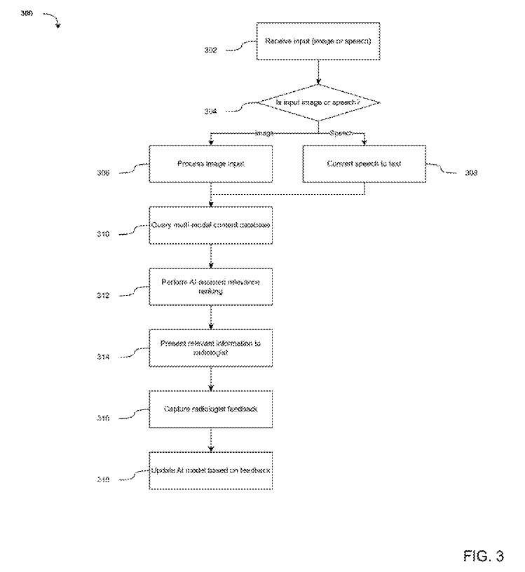

The AI-Assisted Diagnostic Support Companion Product Platform 100 described herein can operate through a structured workflow 300, as illustrated in FIG. 3. This workflow 300 encompasses several key steps, from input reception to AI model updating, designed to provide efficient and accurate diagnostic support for radiologists.

The process begins with step 302, receiving input, which can include an image, speech, or a combination thereof. In some cases, the system 100 can receive medical imaging data such as X-rays, CT scans, or MRI images. In other instances, the input can be in the form of spoken words from a radiologist describing observations or requesting content.

Following input reception (step 302), step 304 can include determining the type of input received. This determination step enables the system 100 to route the input to the appropriate processing module. For image inputs, for example, the system can proceed to step 306 to process the image data. This can involve various image analysis techniques to extract relevant features and content from the medical images, as discussed above. For speech inputs, step 308 can involve converting the speech to text, enabling further processing and analysis of the spoken content.

After processing the input (step 306/ step 308), step 310 can include querying a multi-modal content database. This database can contain a wide range of medical information, including peer-reviewed literature, case studies, and other relevant content. The query is formulated based on the processed input, whether it be extracted image features or converted text from speech.

The system 100 can then employ step 312, AI-assisted relevance ranking to assess and prioritize the retrieved content. This step 312 can utilize machine learning algorithms to determine which pieces of content are most pertinent to the current case. This relevance ranking (step 312) can consider factors such as similarity to the input data, recency of the content, and its applicability to the specific diagnostic context.

Following the relevance ranking (step 312), step 314 can include presenting the most relevant content to the radiologist. This presentation can occur through a user interface designed to integrate seamlessly with the radiologist's workflow. The content can be displayed alongside the original medical images or in a format that allows for easy comparison and reference.

The system can then proceed to step 316 to capture feedback from the radiologist. This feedback can include assessments of the relevance and usefulness of the presented content, as well as any additional insights or observations made by the radiologist. In some cases, this feedback mechanism can enable radiologists to rate the quality of the diagnostic support provided or suggest improvements.

Finally, step 318 can include updating the AI model based on the captured feedback. This step 318 can involve refining the relevance ranking algorithms, adjusting the weighting of various factors in the decision-making process, or expanding the knowledge base with new content derived from the radiologist's input. This continuous learning process can help improve the system's performance over time, potentially leading to more accurate and helpful diagnostic support in future cases.

Through this comprehensive workflow 300, the AI-Assisted Diagnostic Support Companion Product Platform 100 can enhance the diagnostic process, providing radiologists with relevant, timely content while continuously improving its performance based on expert feedback and real-world usage data.

In some implementations, the systems and methods described herein can include and/or can be implemented by one or more specialized computers including specialized hardware and/or software components. For purposes of this disclosure, a specialized computer can be a programmable machine capable of performing arithmetic and/or logical operations and specially programmed to perform the functions described herein, including those performed by the Diagnostic Support Companion Product Platform 100, the AI Engine 102, the Content Matching Engine 208, and other components described throughout this disclosure. In some embodiments, computers can comprise processors, memories, data storage devices, and/or other components. These components can be connected physically or through network or wireless links. Computers can also comprise software which can direct the operations of the components. Computers can be referred to as servers, personal computers (PCs), mobile devices, and other terms for computing/communication devices. For purposes of this disclosure, those terms used herein are interchangeable, and any special purpose computer particularly configured for performing the described functions can be used.

Computers can be linked to one another via one or more networks. A network can be any plurality of completely or partially interconnected computers wherein some or all of the computers are able to communicate with one another. Connections between computers can be wired in some cases (e.g., via wired TCP connection or other wired connection) or can be wireless (e.g., via a Wi-Fi network connection). Any connection through which at least two computers can exchange data can be the basis of a network. Furthermore, separate networks can be able to be interconnected such that one or more computers within one network can communicate with one or more computers in another network. In such a case, the plurality of separate networks can optionally be a single network. In some implementations, the External Systems Interface 112 can facilitate communication between the Diagnostic Support Companion Product Platform 100 and external systems such as the PACS 114 and EMR 116 through such network connections.

The term "computer" shall refer to any electronic device or devices, including those having capabilities to be utilized in connection with an electronic information/transaction system, such as any device capable of receiving, transmitting, processing and/or using data and information. The computer can comprise a server, a processor, a microprocessor, a personal computer, such as a laptop, palm PC, desktop or workstation, a network server, a mainframe, an electronic wired or wireless device, such as for example, a telephone, a cellular telephone, a personal digital assistant, a smartphone, an interactive television, such as for example, a television adapted to be connected to the Internet or an electronic device adapted for use with a television, an electronic pager or any other computing and/or communication device. In some cases, the User Interface 110 can be implemented on such devices to enable radiologists to interact with the system 100.

The term "network" shall refer to any type of network or networks, including those capable of being utilized in connection with the systems and methods described herein, such as, for example, any public and/or private networks, including, for instance, the Internet, an intranet, or an extranet, any wired or wireless networks or combinations thereof.

A computer-readable storage medium is contemplated by the present disclosure. The computer-readable storage medium includes one or more sequences of computer-readable instructions that, when executed by one or more processors, cause a computer device, system and/or platform to perform any of the operations described herein, including those described as being performed by the Diagnostic Support Companion Product Platform 100, the AI Engine 102, the Speech Recognition Module 106, the Image Processing Module 108, the Content Matching Engine 208, the Content Retrieval Module 214, and/or related components, devices and/or systems of the present disclosure.

The term "computer-readable storage medium" should be taken to include a single medium or multiple media that store one or more sets of instructions. The term "computer-readable storage medium" shall also be taken to include any medium that can store or encode a set of instructions for execution by the machine and that causes the machine to perform any one or more of the methodologies of the present disclosure, including the workflow 300 illustrated in FIG. 3.

While the present disclosure has been discussed in terms of certain embodiments, it should be appreciated that the present disclosure is not so limited. The embodiments are explained herein by way of example, and there are numerous modifications, variations and other embodiments that can be employed that would still be within the scope of the present disclosure.

A number of implementations have been described. Nevertheless, it will be understood that various modifications may be made without departing from the spirit and scope of the disclosure. Accordingly, other implementations are within the scope of the following claims.

What is claimed is:

1. A diagnostic support system for radiologists, comprising:
a user interface configured to receive user input and present relevant content to the user, the user input comprising at least one of medical imaging data and speech input;
an AI engine configured to process medical imaging data;
a content matching engine configured to match the user input with the relevant content, the content matching engine comprising a relevance determinator and a neural network;
a content retrieval module configured to receive matched content identifiers from the content matching engine and retrieve content from a multi-modal content database, the multi-modal content database storing peer-reviewed content resources;
a speech recognition module configured to convert speech input to text;
an image processing module configured to analyze medical images; and
an external systems interface configured to communicate with hospital information systems,
wherein the relevance determinator assesses a significance and applicability of the retrieved content,
the neural network recognizes patterns and makes determination as to a relevance of the retrieved content,
the AI engine combines outputs from the relevance determinator and the neural network to produce a final set of matched content from among the retrieved content, and
the final set of matched content comprising real-time diagnostic support presented to the user via the user interface.

2. The diagnostic support system of claim 1, wherein the AI engine employs a hybrid approach combining computer vision, neural networks, and generative AI techniques for content matching and retrieval.

3. The diagnostic support system of claim 1, wherein the multi-modal content database stores medical journal articles, case studies, and imaging reference materials.

4. The diagnostic support system of claim 3, wherein the multi-modal content database is periodically updated to incorporate additional journal issues and published research.

5. The diagnostic support system of claim 1, wherein the speech recognition module employs natural language processing algorithms to accurately interpret medical terminology and diverse speech patterns.

6. The diagnostic support system of claim 1, wherein the image processing module is configured to process multiple imaging modalities including X-ray, computed tomography (CT), and magnetic resonance imaging (MRI).

7. The diagnostic support system of claim 6, wherein the image processing module employs deep learning neural networks trained on large datasets of medical images to recognize patterns, anomalies, and specific features within the images.

8. The diagnostic support system of claim 1, wherein the user interface is configured to display a dashboard presenting an overview of current cases and highlighting those requiring immediate attention.

9. The diagnostic support system of claim 1, wherein the external systems interface utilizes standard healthcare data exchange protocols to ensure compatibility with a wide range of hospital systems.

10. The diagnostic support system of claim 9, wherein the external systems interface enables bidirectional communication with Picture Archiving and Communication Systems (PACS) and retrieves relevant patient data from Electronic Medical Record (EMR) systems.

11. A method for providing diagnostic support to radiologists, comprising:
receiving input data comprising at least one of medical imaging data or speech input;
processing the input data using an AI engine;
querying a multi-modal content database storing peer-reviewed content resources based on the processed input data;
performing AI-assisted relevance ranking on query results, comprising:
performing an initial assessment of potential content matches using a relevance determinator based on predefined criteria,
conducting an in-depth analysis of the input data and the potential content matches using a neural network, and
combining results from the relevance determinator and the neural network to produce a ranked set of matched content;
presenting the ranked set of matched content to a radiologist through a user interface;
capturing feedback from the radiologist; and
updating an AI model based on the captured feedback.

12. The method of claim 11, wherein processing the input data comprises:
converting speech input to text using natural language processing algorithms configured to interpret medical terminology; and
analyzing medical imaging data using deep learning neural networks trained on large datasets of medical images.

13. The method of claim 12, wherein analyzing medical imaging data comprises identifying and highlighting potential areas of concern within the medical images.

14. The method of claim 11, wherein querying the multi-modal content database comprises:
formulating a query based on extracted features from medical imaging data or converted text from speech input; and
employing advanced database querying techniques to efficiently access and extract relevant content.

15. The method of claim 14, wherein the multi-modal content database stores medical journal articles, case studies, and imaging reference materials.

16. The method of claim 15, further comprising periodically updating the multi-modal content database to incorporate journal issues and published research.

17. The method of claim 11, wherein performing AI-assisted relevance ranking comprises:
assessing retrieved content using machine learning algorithms; and
prioritizing content based on similarity to input data, recency, and applicability to the specific diagnostic context.

18. The method of claim 17, wherein presenting relevant content to the radiologist comprises displaying the content alongside original medical images in a user interface designed to integrate with the radiologist's workflow.

19. The method of claim 18, wherein capturing feedback from the radiologist comprises:
receiving assessments of the relevance and usefulness of the presented content; and
collecting additional insights or observations made by the radiologist.

20. The method of claim 19, wherein updating the AI model based on the captured feedback comprises:
refining relevance ranking algorithms;
adjusting weighting of factors in the decision-making process; and
expanding a knowledge base with new content derived from the radiologist's input.

21. The method of claim 11, further comprising:
detecting, using the AI engine, one or more anomalies in the medical imaging data;
determining that at least one detected anomaly corresponds to a potential diagnostic finding requiring immediate attention;
generating an alert identifying the potential diagnostic finding; and
automatically prioritizing a case in a radiologist's workflow queue based on the at least one detected anomaly.

22. The method of claim 11, wherein processing the input data comprises:
converting the medical imaging data into a feature representation using an image processing neural network;
generating embedding vectors from the feature representation, wherein the embedding vectors represent semantic characteristics of the medical imaging data;
partitioning the embedding vectors into clusters corresponding to different diagnostic categories;
matching the clusters to content entries in the multi-modal content database; and
synthesizing a content presentation comprising content entries corresponding to matched clusters, wherein the content presentation is tailored to the specific diagnostic context of the medical imaging data.
